(19) United States
(12) Patent Application Publication
(10) Pub. No.: US 2026/0276613 A1
(43) Pub. Date: Sep. 17, 2026

(54) LABORATORY MONITORING DEVICES AND SYSTEMS

(71) Applicants: Laurin Oberhauser, Schaanwald (LI); Adrian Wegiel, Schaanwald (LI)

(72) Inventors: Laurin Oberhauser, Schaanwald (LI); Adrian Wegiel, Schaanwald (LI)

(21) Appl. No.: 19/078,281

(22) Filed: Mar. 13, 2025

(51) Int. Cl. G01N 33/00; G01D 21/02; H04L 41/5074

(52) U.S. Cl. CPC G01N 33/0063; G01D 21/02; G01N 33/004; G01N 33/0075; H04L 41/5074

(57) ABSTRACT

An embodiment of the present invention is a laboratory environmental monitoring system, comprising: a gateway; and one or more nodes having at least one environmental sensor, wherein: the gateway is connected to a cloud-based internet application by a 3 stage internet solution (LAN, Wifi, Sim card); and the one or more nodes are connected to the gateway by a Low Power Wide Area Network.

### BACKGROUND

Devices and systems for environmental monitoring have been described in the prior art patent literature.

For example, U.S. Pat. No. 10,545,038 discloses that an electronic device may be provided with environmental sensors. Environmental sensors may include one or more environmental sensor components and one or more acoustic components. Acoustic components may include a speaker or a microphone. Environmental sensor components may include a temperature sensor, a pressure sensor, a humidity sensor, a gas sensor, or other sensors or combinations of sensors for sensing attributes of the environment surrounding the device. The environmental sensor may have an enclosure with an opening. The enclosure may be formed from a rigid support structure and a portion of a printed circuit. The opening may be formed in the rigid support structure or the printed circuit. The opening in the enclosure for the environmental sensor may be aligned with an opening in an outer structural member for the device. The outer structural member may be a housing structure or a cover layer for a device display.

US20230208919 discloses environmental monitoring systems that include base stations and environmental sensor units (ESUs) that communicate in a wireless sensor network. A base station may include environmental sensors and may communicate with a remote server using a wide-area wireless network. The base station may also communicate with an environmental sensor unit using a wireless personal area network. The ESU may include environmental sensors and may communicate with the remote server via the base station. The base station and ESU may be adapted to automatically pair with one another to enable the ESU to communicate via the base station. An ESU may also be adapted to process data regarding environmental conditions by automatically determining a type of environmental sensor from which the data was received and processing the data based on the type. An ESU may also include physical ports by which new sensors can be connected to the ESU.

However, prior art systems, methods, and devices are lacking in reliability and consume large amounts of bandwidth, among other shortcomings.

### SUMMARY OF INVENTION

Therefore, the present invention provides devices and systems for monitoring of environmental conditions, particularly conditions in a laboratory setting. A gateway hub device displays status, maintenance information, and alarms relating to one or more connected node devices comprising environmental and energy monitoring sensors. The gateway hub connects to a cloud-based internet application by a network connection and the node devices connect to the gateway hub using a minimum of bandwidth, avoiding overload of the laboratory internet network.

According to an aspect of the present invention, there is provided a laboratory environmental monitoring system, comprising: a gateway; and one or more nodes having at least one environmental or energy monitoring sensor, wherein: the gateway is connected to a cloud-based internet application by a Local Area Network; and the one or more nodes are connected to the gateway by a Low Power Wide Area Network.

### BRIEF DESCRIPTION OF THE DRAWINGS

FIG. 1 illustrates a laboratory monitoring system including a gateway device according to an embodiment of the present invention.

FIG. 2 illustrates a node in the laboratory monitoring system according to an embodiment of the present invention.

FIG. 3 illustrates another view of the node in the laboratory monitoring system according to an embodiment of the present invention.

FIG. 4A illustrates the components of a node in an alternate embodiment.

FIG. 4B illustrates the node in the alternate embodiment with its cover.

FIG. 5 illustrates the back of the node in the alternate embodiment.

FIG. 6 illustrates the gateway device.

### DETAILED DESCRIPTION

In embodiments of the present invention, a gateway device serves as the data receiver for all information sent from connected node devices.

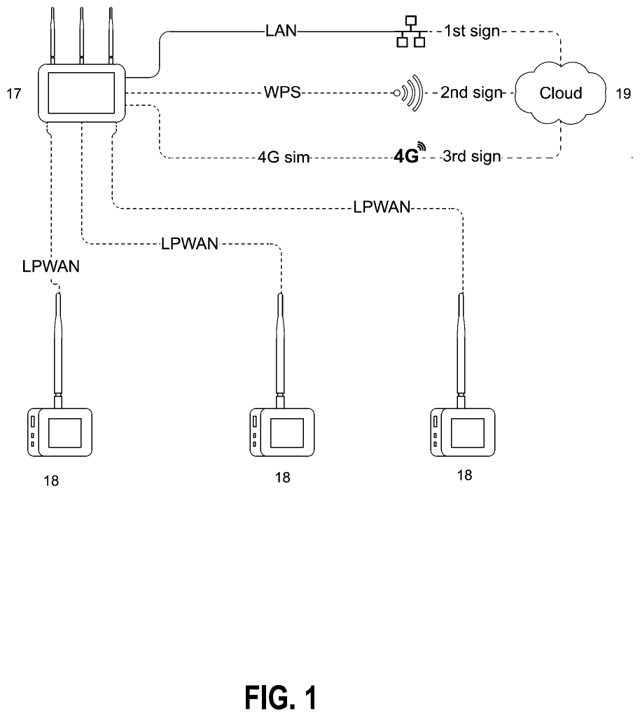

FIG. 1 illustrates a laboratory monitoring system including a gateway device according to an embodiment of the present invention.

The node devices 18 connect to the gateway device 17 by LPWAN, “Low Power Wide Area Network”, a kind of wireless telecommunication vast area network that engineers long-range communications at a low bit rate. LPWAN can be LoRaWAN®. Node devices 18 do not use WiFi so as to not overload the WiFi bandwidth available in the location, e.g., the laboratory.

The gateway device 17 connects to the cloud 19 by a network internet connection, i.e., a LAN connection. A WiFi connection can be used when LAN connection is not available. A preferred embodiment also has a SIM card for backup communications when the primary LAN connection and WPS connection are inoperative.

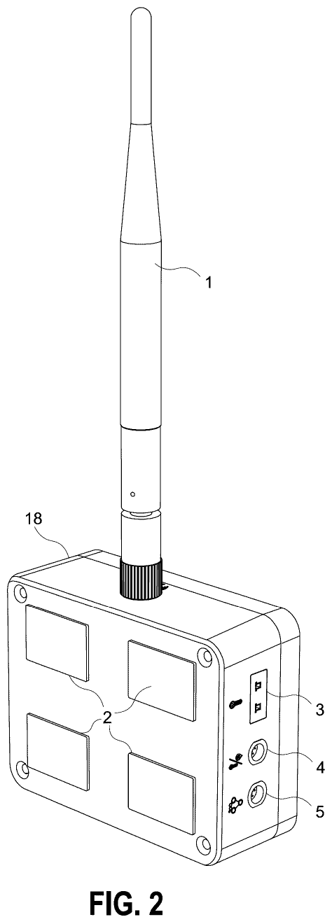

FIG. 2 illustrates a node in the laboratory monitoring system according to an embodiment of the present invention.

Node devices use antenna 1 to send data to the gateway device associated with the node. The node devices 18 continuously measure data with internal sensors and buffer when there is a loss of communication and send data gathered during an outage when communication is restored.

Magnets 2 can be provided with node device 18 for ease in attaching node device 18 to various metallic surfaces and retaining node device 18 in position.

Node devices 18 can be provided with sensors for monitoring many custom parameters.

Node devices 18 can be provided for example with thermocouple sensor port 3. Sensors on port 3 can operate under a variety of temperature conditions and measure changes in temperature. Thermocouple sensor 3 only measures temperature and is an analog sensor which can measure temperatures down to −200 degrees Celsius (a digital sensor can only measure down to about −40 degrees Celsius).

Node devices 18 can be provided with a combination temperature and humidity sensor port 4. Sensor on port 4 can operate under a variety of humidity conditions ranging from 0 to 100% humidity/−40° C. to +60° C. and measure changes in humidity and temperature within the entire range.

A Bluetooth temperature/humidity combi sensor can also connect directly to a microchip provided within node device 18.

The temperature and/or humidity sensors can be positioned remotely from node devices 18 and can communicate by BLE with node devices 18. The remote sensors have lithium batteries that last at least one year. The Node devices 18 can have LCD screens displaying temperature, humidity, Co2 and other values.

Node devices 18 can be provided with sensor 5 for measuring the concentration of carbon dioxide, oxygen, or other gases. Sensor port 5 uses RS-485 and can connect to a variety of sensors. For example, node devices 18 can also measure ambient pressure. Sensor ports 4 and 5 are identical except for the icon used to label them.

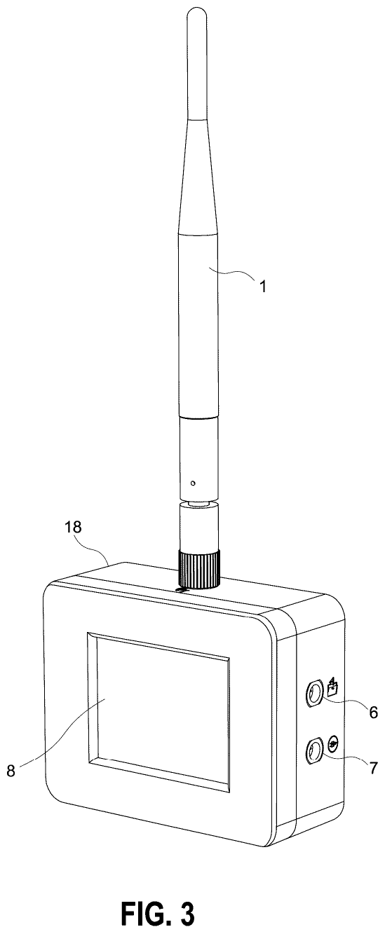

FIG. 3 illustrates another view of the node in the laboratory monitoring system according to an embodiment of the present invention.

Node devices 18 can be provided with display screen 8 which can be an LED display. The display screen 8 displays the measurements collected by the node device 18 and transmitted to the gateway by antenna 1. The display also displays the signal strength it has to the gateway.

Node device 18 has a power port 7 to connect to a power supply, i.e., an outlet.

Node devices 18 can be provided with a sensor port 6 to measure door opening and closing. The sensors can be provided on the door as well as on the node and a processor can calculate based on the positions whether the door is open or closed. A lock can be provided in combination with a sensor and a password can be entered on the node device to open the lock.

A variety of other custom environmental sensors can be provided in node devices 18 specific to the particular site. Node devices 18 can be provided for example with a fluid level sensor connected to fluid level sensor port 6. The sensor can detect if fluid falls below a set threshold.

Node device 18 automatically detects which type of sensor it is connected to and sends the appropriate data string to the gateway based on the type of sensor.

The main PCB is adaptable with different adapter PCBs to a light intensity sensor, pressure sensor, a thermocouple sensor, and a temperature/humidity combo sensor. The node device 18 can read multiples temperatures at the same time along with pressure and at least one custom sensor.

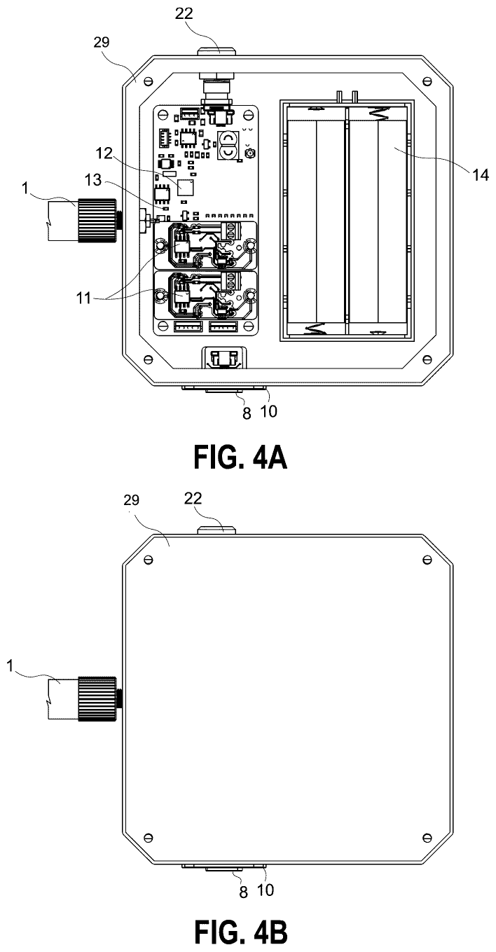

FIG. 4A illustrates the components of a node in an alternate embodiment.

Node device 29 is a lite node device. Multiple types of node devices are possible. Standard node devices are powered by an external power supply, i.e., they plug into an outlet. Lite node devices are powered by an internal rechargeable battery, battery 14. The batteries are configured to last at least 12 months without recharging. The batteries can be LiFePO4; more than one battery can be provided for each node. The batteries and the node device can withstand extreme temperatures, between −20 C and 60 C.

Antenna 1 is the same antenna as the antenna in the node device described above.

The lite node has adapter PCBs 11. On the main PCB, there are two slots to plug in adapter PCBs. These adapter PCBs have different sensor configurations (thermocouple, pressure, light intensity, door openings, I2C communication for external temperature, humidity, pressure, and Co2 combination sensor).

Temperature/humidity combi sensor 13 is onboard the PCB on the node lite, whereas on the regular node the combi sensor is an external sensor which can connect. Pressure sensor 12 is onboard the PCB on the node lite.

Port 8 is a service (programming) port and port 10 is a charging port. The service port can be used to upload new firmware. Port 10 can also be used as a serial communication port to see the device logging.

FIG. 4B illustrates the node in the alternate embodiment with its cover.

Lite node device 29 is shown with the cover on and antenna 1 coming from lite node device 29. Port 8 is a service (programming) port and port 10 is a charging port.

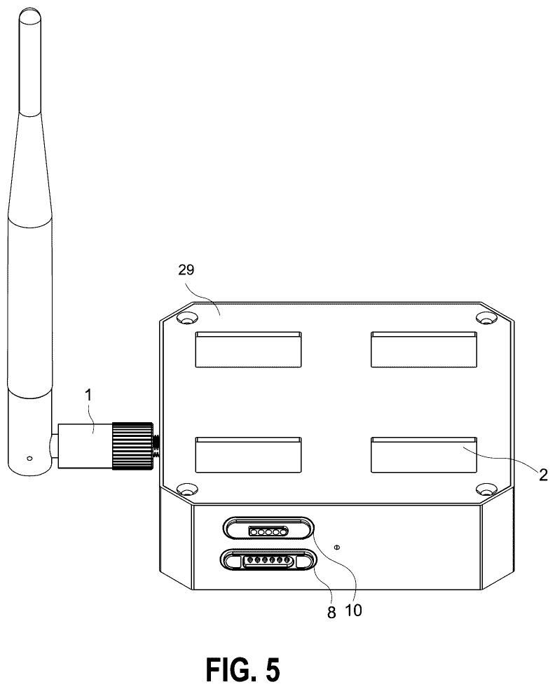

FIG. 5 illustrates the back of the node in the alternate embodiment.

Lite node device 29 is shown with the cover on and antenna 1 coming from lite node device 29. Ventilation slots allow for the pressure sensor and temperature/humidity sensor to expose to the outer environment. Magnets 2 can be used to mount the lite node device to a metal surface such as a refrigerator. Port 8 is a service (programming) port and port 10 is a charging port.

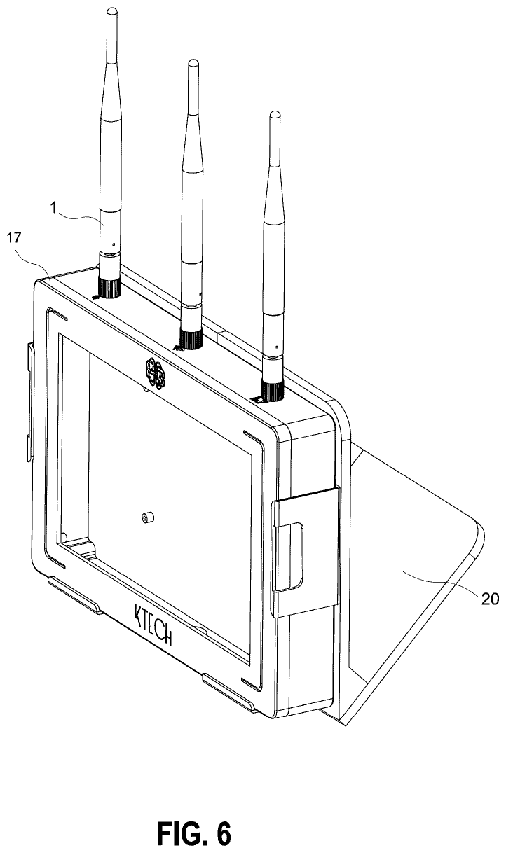

FIG. 6 illustrates the gateway device.

The gateway device 17 has antennas 1 and stand 20. Multiple antennas are featured; the first antenna is for LoRa communication to the gateway, the middle antenna is for WiFi, and third antenna is for a SIM card.

The gateway device 17 displays information such as sensor data sent from connected node devices 18 on an LCD screen provided on gateway device 17.

The gateway device 17 displays an indication of the internet status (LAN, WI-FI or 4G SIM) based on which connection is operable and being utilized at a given time. The gateway device 17 can search for available Wi-Fi networks.

The gateway device 17 displays preventive maintenance information, such as countdowns to scheduled and/or recommended maintenance intervals. The gateway device 17 can display open support tickets, for example.

The gateway device 17 displays the status of node devices 18, such as online or offline. The gateway device 17 can display active alarms based on data points measured by sensors associated with node devices 18 exceeding predefined thresholds.

The gateway device 17 is provided with an LED status indicator that can change colors to indicate status of operation, such as normal operation or error.

The gateway device 17 is powered by an external power supply, i.e., by plugging into an outlet.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A laboratory environmental monitoring system, comprising:
a gateway; and
one or more nodes having one or more environmental or power monitoring sensors, wherein:
the gateway is connected to a cloud-based internet application by a Local Area Network; and
the one or more nodes are connected to the gateway by a Low Power Wide Area Network.

2. The laboratory environmental monitoring system of claim 1, wherein the gateway is provided with an LCD screen displaying statuses of the one or more nodes.

3. The laboratory environmental monitoring system of claim 1, wherein one or more of the one or more environmental sensors is a temperature sensor.

4. The laboratory environmental monitoring system of claim 1, wherein one or more of the one or more environmental sensors is a humidity sensor.

5. The laboratory environmental monitoring system of claim 3, wherein the temperature sensor is remote from the node device.

6. The laboratory environmental monitoring system of claim 1, wherein one or more of the one or more environmental sensors is a proximity sensor.

7. The laboratory environmental monitoring system of claim 6, wherein there is a proximity sensors with one provided on a door that connects to the node and a processor in the gateway devices calculates based on the positions whether the door is open or closed.

8. The laboratory environmental monitoring system of claim 1, wherein one or more of the one or more environmental sensors is a fluid level sensor configured to detect if a fluid level falls below a set threshold.

9. The laboratory environmental monitoring system of claim 1, wherein one or more of the one or more environmental sensors is a sensor for measuring the concentration of carbon dioxide, oxygen, or another gas.

10. The laboratory environmental monitoring system of claim 1, wherein the one or more nodes buffer when there is a loss of communication and send data gathered during the loss of communication when communication is restored.

11. The laboratory environmental monitoring system of claim 1, wherein the gateway device displays active alarms based on data points measured by the sensors associated with the node devices exceeding predefined thresholds.

12. The laboratory environmental monitoring system of claim 1, wherein the gateway device displays countdowns to scheduled and/or recommended maintenance intervals.

13. The laboratory environmental monitoring system of claim 1, wherein the gateway device is provided with a SIM card.

14. The laboratory environmental monitoring system of claim 1, wherein the gateway device displays open support tickets.

15. The laboratory environmental monitoring system of claim 1, wherein the gateway device is configured to search for available Wi-Fi networks.

16. The laboratory environmental monitoring system of claim 13, wherein the gateway device displays an indication of the internet status (LAN, WI-FI or SIM) based on which connection is operable and being utilized at a given time.

17. The laboratory environmental monitoring system of claim 5, wherein the temperature sensor has an internal coin battery.

18. The laboratory environmental monitoring system of claim 4, wherein the humidity sensor is remote from the node device.

19. The laboratory environmental monitoring system of claim 18, wherein the temperature/humidity sensor has an internal coin battery.

20. The laboratory environmental monitoring system of claim 1, wherein the one or more node devices have LCD screens displaying status information.
